(19) United States
(12) Patent Application Publication
(10) Pub. No.: US 2018/0085415 A1
(43) Pub. Date: Mar. 29, 2018

(54) METHOD OF TREATING MACROPHAGE FOAM CELL FORMATION AND DISEASES ASSOCIATED WITH MACROPHAGE FOAM CELL FORMATION

(71) Applicant: Board of Supervisors of Louisiana State University and Agricultural and Mechanical College, Baton Rouge, LA (US)

(72) Inventors: James Allen CARDELLI, Shreveport, LA (US); Matthew Dale WOOLARD, Shreveport, LA (US)

(21) Appl. No.: 15/712,958

(22) Filed: Sep. 22, 2017

Related U.S. Application Data
(60) Provisional application No. 62/398,823, filed on Sep. 23, 2016.

(51) Int. Cl. A61K 36/282; A61K 36/78; A61K 36/58; A61K 31/352

(52) U.S. Cl. CPC A61K 36/282; A61K 31/352; A61K 36/58; A61K 36/78

(57) ABSTRACT

Products and methods of treating a foam cell associated disease comprising administering a pharmaceutical composition including a therapeutically effective amount of a first therapeutic; wherein the first therapeutic is one of an extract of Artemisia dracunculus (Russian Tarragon) leaves, an extract of Melia azedarach (Chinaberry) leaves, an extract of Saururus cernuus (Lizard's Tail) Roots, and an extract of Sambucus Canadensis (Elderberry) leaves, or pharmacologically acceptable salts, solvates, esters, amides, clathrates, stereoisomers, enantiomers, prodrugs or analogs thereof, or a combination thereof.

### CROSS REFERENCE TO RELATED APPLICATIONS/PRIORITY

The present invention claims priority to United States Provisional Patent Application No. 62/398,823, filed Sep. 23, 2016, which is incorporated by reference into the present disclosure as if fully restated herein. Any conflict between any incorporated material and the specific teachings of this disclosure shall be resolved in favor of the latter. Likewise, any conflict between an art-understood definition of a word or phrase and a definition of the word or phrase as specifically taught in this disclosure shall be resolved in favor of the latter.

### STATEMENT REGARDING FEDERALLY SPONSORED RESEARCH

The inventors received support through from NIH grant project number 2P50AT002776-11, titled CEFALU: Overall; Botanicals and metabolic resiliency (CEFALU).

### FIELD OF THE INVENTION

The invention generally relates to the method and use of extracts of plants growing in Louisiana and other places in the treatment of diseases including cardiovascular disease and kidney disease, and to the prevention and reversal of macrophage foam cell formation. The foam cell plays a major role in atherosclerosis.

### BACKGROUND OF THE INVENTION

The prevalence of both type II diabetes and the pre-diabetic condition known as the metabolic syndrome (MetS) has increased in recent years. MetS is the presence of co-existing risk factors such as hypertension, dislipidemia, glucose intolerance and insulin resistance; all of which increase the risk of cardiovascular disease (CVD), the number one comorbidity of MetS. The public health impact of metabolic syndrome is substantial and successful strategies to reduce the metabolic syndrome burden are needed. Current research is aimed at developing therapeutic strategies to increase insulin sensitivity and reduce inflammatory processes.

The most prominent underlying cause of CVD is atherosclerosis, and type II diabetes and MetS both enhance the progression of atherosclerosis. Atherosclerosis is a chronic inflammatory condition of small and medium sized arteries initiated by dyslipidemia and mediated by macrophages. Hyperlipidemia promotes the deposition of cholesterol-rich low density lipoprotein (LDL) into the arterial intima, leading to the recruitment of monocyte-derived macrophages. Macrophages are required for the recycling of LDL-derived cholesterol as part of the reverse cholesterol transport system. LDL becomes modified (e.g. oxidized-LDL) during prolonged states of intimal retention. Modification of LDL enhances its uptake by macrophages and inhibits reverse cholesterol transport (RCT), resulting in lipid-laden foam cells. Through mechanisms that are still enigmatic, macrophage foam cells begin to release pro-inflammatory factors that contribute to atheroma formation, plaque progression, and plaque instability leading to the catastrophic events associated with CVD. Current therapies to treat CVD, even in individuals with MetS, attempt to lower serum levels of LDL cholesterol, yet this only results in a 30% reduction in catastrophic CVD events. Current therapies fail address the therapeutically advantageous strategy of targeting plaque intrinsic processes such as foam cell formation and inflammatory responses.

While some aspects of extracts derived from Artemisia dracunculus (Russian Tarragon or “RT”) leaves, Melia azedarach (Chinaberry or CB) leaves, Saururus cernuus (Lizard's Tail or “LT”) Roots and Sambucus Canadensis (Elderberry or “EB”) leaves, have been studied, it was unknown if these extracts can alter macrophage processes that mediate atherosclerosis and ultimately CVD. In the disclosed experiments, the inventors used an in vitro model of foam cell formation to investigate capability of these compounds to reduce foam cell formation and foam cell inflammatory responses.

### SUMMARY OF THE INVENTION

Wherefore, it is an object of the present invention to overcome the above mentioned shortcomings and drawbacks associated with the prior art.

Metabolic syndrome (MetS) is a cluster of risk factors (diabetes, prediabetes, abdominal obesity, high cholesterol and high blood pressure) that significantly contribute to cardiovascular disease (CVD), the number one killer of Americans. As such, CVD is the most significant comorbidity associated with MetS and diabetes. MetS accelerates the process of atherosclerosis which is a cholesterol-driven chronic inflammatory complication of large and medium size arteries, mediated by macrophages. Deposition of cholesterol, in the form of low density lipoproteins (LDLs), into the arterial intima, drives the influx of macrophages to clear these lipids, as part of reverse cholesterol transport (RCT). However, prolonged elevation of serum LDLs, as observed during MetS, leads to the formation of modified LDLs in the arterial intima (mod-LDLs). Modification of LDLs results in unregulated uptake of LDLs and reduced efflux of cholesterol from macrophages causing a lipid laden phenotype called foam cells. These foam cells eventually release pro-inflammatory mediators. The combination of excessive mod-LDL uptake, reduced cholesterol efflux, and pro-inflammatory mediator production promotes atheroma formation, plaque progression, and ultimately plaque instability, culminating in the catastrophic events associated with CVD, such as heart attacks and stroke. Current therapies to reduce CVD in patients, including those with MetS, is to reduce serum levels of cholesterol, but this only reduces deaths by catastrophic CVD by 30%. To further reduce mortality associated with CVD, the inventors realized that targeting the processes that are ongoing in the plaque would be advantageous.

The present invention involves the discovery that certain plant extracts can reverse macrophage foam cell formation and prevent macrophage foam cell formation in situations where macrophage foam cells would otherwise form. Russian Tarragon (RT) and Chinaberry (CB) leaf extract reduced the formation of Nile Red positive lipid inclusions induced by oxidized LDL. RT but not CB reduced the uptake of oxLDL. Both extracts stimulated cholesterol efflux and reversed the macrophage foam cell phenotype. RT reduced the production of pro inflammatory cytokines by lipid laden macrophages. Thus, based on the disclosed experiments, these extracts alone, in combination with each other or in combination with select flavonoids could be used to prevent and reverse formation of macrophage foam cells thus blocking a key event in the generation of atherosclerotic plaques, resulting in a reduction in heart attacks and strokes.

The present invention relates to pharmaceutical compositions of a therapeutic (e.g., RT leaves extract, CB leaves extract, LT roots extract, and EB leaves extract), or a pharmaceutically acceptable salt, solvate, or prodrug thereof, and use of these compositions for the treatment of a foam cell associated disease, including kidney disease, cancer, metabolic syndrome (MetS), atherosclerosis, cardiovascular disease (CVD), tuberculosis, Parkinson's disease, a neurological disorder, and a disease where lipid droplet formation and autophagic flux play a role, or a pre-disease state thereof.

The invention is related to products and methods of treating a foam cell associated disease comprising administering a pharmaceutical composition including a therapeutically effective amount of a first therapeutic; wherein the first therapeutic is one of an extract of Artemisia dracunculus (Russian Tarragon) leaves, an extract of Melia azedarach (Chinaberry) leaves, an extract of Saururus cernuus (Lizard's Tail) Roots, and an extract of Sambucus Canadensis (Elderberry) leaves, or pharmacologically acceptable salts, solvates, esters, amides, clathrates, stereoisomers, enantiomers, prodrugs or analogs thereof, or a combination thereof. According to a further embodiment, the foam cell associated disease is one of kidney disease, cancer, metabolic syndrome (MetS), atherosclerosis, cardiovascular disease (CVD), tuberculosis, Parkinson's disease, a neurological disorder, and a disease where lipid droplet formation and autophagic flux play a role, or a pre-disease state thereof. According to a further embodiment, the first therapeutic includes both an extract of Russian Tarragon leaves and an extract of Chinaberry leaves or pharmacologically acceptable salts, solvates, esters, amides, clathrates, stereoisomers, enantiomers, prodrugs or analogs thereof, or a combination thereof. According to a further embodiment, the pharmaceutical composition further includes a therapeutically effective amount of a second therapeutic, distinct from the first therapeutic. According to a further embodiment, the second therapeutic is a polyphenols. According to a further embodiment, the polyphenol is a flavonoid. According to a further embodiment, the second therapeutic is one of epigallocatechin-3-gallate (EGCG), luteolin, quercetin, and ellagic acid.

The invention is further related to products and methods of treating an inflammation associated disease comprising administering a pharmaceutical composition including a therapeutically effective amount of a first therapeutic; wherein the first therapeutic is one of an extract of Artemisia dracunculus (Russian Tarragon) leaves, an extract of Melia azedarach (Chinaberry) leaves, an extract of Saururus cernuus (Lizard's Tail) Roots, and an extract of Sambucus Canadensis (Elderberry) leaves, or pharmacologically acceptable salts, solvates, esters, amides, clathrates, stereoisomers, enantiomers, prodrugs or analogs thereof, or a combination thereof. According to a further embodiment, the pharmaceutical composition further comprises a therapeutically effective amount of a second therapeutic distinct from the first therapeutic and wherein the second therapeutic is an anti-inflammation therapeutic. According to a further embodiment, the inflammation associated disease is one of asthma, arthritis, Crohn's disease, Alzheimer's disease, cancer, cardiovascular disease, diabetes, high blood pressure, high cholesterol levels, Parkinson's disease, metabolic syndrome (MetS), atherosclerosis, or a pre-disease state thereof.

The invention is further related to methods and therapeutic products comprising a first pharmaceutically active agent being one of one of an extract of Artemisia dracunculus (Russian Tarragon) leaves, an extract of Melia azedarach (Chinaberry) leaves, an extract of Saururus cernuus (Lizard's Tail) Roots, and an extract of Sambucus Canadensis (Elderberry) leaves, or pharmacologically acceptable salts, solvates, esters, amides, clathrates, stereoisomers, enantiomers, prodrugs or analogs thereof, or a combination thereof; and a second pharmaceutically active agent being one of one of an extract of Russian Tarragon leaves, an extract of Chinaberry leaves, an extract of Lizard's Tail Roots, an extract of Elderberry leaves, and a polyphenol, or pharmacologically acceptable salts, solvates, esters, amides, clathrates, stereoisomers, enantiomers, prodrugs or analogs thereof, or a combination thereof; wherein the first pharmaceutically active agent is chemically distinct from the second pharmaceutically active agent. According to a further embodiment, the first pharmaceutically active agent is extract of Russian Tarragon leaves or a pharmacologically acceptable salt, solvate, ester, amide, clathrate, stereoisomer, enantiomer, prodrug or analog thereof, or a combination thereof. According to a further embodiment, the first pharmaceutically active agent is on of an extract of Russian Tarragon leaves a pharmacologically acceptable salt, solvate, ester, amide, clathrate, stereoisomer, enantiomer, prodrug or analog thereof, or a combination thereof and the second pharmaceutically active agent is one of an extract of Chinaberry leaves a pharmacologically acceptable salt, solvate, ester, amide, clathrate, stereoisomer, enantiomer, prodrug or analog thereof, or a combination thereof. According to a further embodiment, the second pharmaceutically active agent is one of a polyphenol, a pharmacologically acceptable salt, solvate, ester, amide, clathrate, stereoisomer, enantiomer, prodrug or analog thereof, or a combination thereof. According to a further embodiment, the second pharmaceutically active agent is one of a flavonoid, a pharmacologically acceptable salt, solvate, ester, amide, clathrate, stereoisomer, enantiomer, prodrug or analog thereof, or a combination thereof. According to a further embodiment, the second pharmaceutically active agent is one of pigallocatechin-3-gallate (EGCG), luteolin, quercetin, ellagic acid, pharmacologically acceptable salts, solvates, esters, amides, clathrates, stereoisomers, enantiomers, prodrugs or analogs thereof, or a combination thereof. According to a further embodiment, a third pharmaceutically active agent is included with is one of one of an extract of Russian Tarragon leaves, an extract of Chinaberry leaves, an extract of Lizard's Tail Roots, an extract of Elderberry leaves, and a polyphenol, or pharmacologically acceptable salts, solvates, esters, amides, clathrates, stereoisomers, enantiomers, prodrugs or analogs thereof, or a combination thereof. According to a further embodiment, the first, the second, and the third pharmaceutically active agents are each extracts from different plants, or pharmacologically acceptable salts, solvates, esters, amides, clathrates, stereoisomers, enantiomers, prodrugs or analogs thereof, or a combination thereof. According to a further embodiment, the first and the second pharmaceutically active agents are each extracts from different plants, or pharmacologically acceptable salts, solvates, esters, amides, clathrates, stereoisomers, enantiomers, prodrugs or analogs thereof, or a combination thereof, and third pharmaceutically active agent is a polyphenol or a pharmacologically acceptable salt, solvate, ester, amide, clathrate, stereoisomer, enantiomer, prodrug or analog thereof, or a combination thereof. According to a further embodiment, the first pharmaceutically active agent is an extract from a plant, or a pharmacologically acceptable salt, solvate, ester, amide, clathrate, stereoisomer, enantiomer, prodrug or analog thereof, or a combination thereof, and the second and the third pharmaceutically active agent is a polyphenol or pharmacologically acceptable salts, solvates, esters, amides, clathrates, stereoisomers, enantiomers, prodrugs or analogs thereof, or a combination thereof.

In some embodiments, the therapeutic, or a pharmaceutically acceptable salt, solvate, or prodrug thereof, is administered as a pharmaceutical composition that further includes a pharmaceutically acceptable excipient.

In some embodiments, administration of the pharmaceutical composition to a human results in a peak plasma concentration of the therapeutic between 0.05 μM-10 μM (e.g., between 0.05 μM-5 μM).

In some embodiments, the peak plasma concentration of the therapeutic is maintained for up to 14 hours. In other embodiments, the peak plasma concentration of the therapeutic is maintained for up to 1 hour.

In some embodiments, the condition is a foam cell associated disease.

In certain embodiments, the foam cell associated disease is mild to moderate foam cell associated disease.

In further embodiments, the foam cell associated disease is moderate to severe foam cell associated disease.

In other embodiments, the therapeutic is administered at a dose that is between 0.05 mg-5 mg/kg weight of the human.

In certain embodiments, the pharmaceutical composition is formulated for oral administration.

In other embodiments, the pharmaceutical composition is formulated for extended release.

In still other embodiments, the pharmaceutical composition is formulated for immediate release.

In some embodiments, the pharmaceutical composition is administered concurrently with one or more additional therapeutic agents for the treatment or prevention of the foam cell associated disease.

In some embodiments, the therapeutic, or a pharmaceutically acceptable salt, solvate, or prodrug thereof, is administered as a pharmaceutical composition that further includes a pharmaceutically acceptable excipient.

In some embodiments, administration of the pharmaceutical composition to a human results in a peak plasma concentration of the therapeutic between 0.05 μM-10 μM (e.g., between 0.05 μM-5 μM).

In some embodiments, the peak plasma concentration of the therapeutic is maintained for up to 14 hours. In other embodiments, the peak plasma concentration of the therapeutic is maintained for up to 1 hour.

In other embodiments, the therapeutic is administered at a dose that is between 0.05 mg-5 mg/kg weight of the human.

In certain embodiments, the pharmaceutical composition is formulated for oral administration.

In other embodiments, the pharmaceutical composition is formulated for extended release.

In still other embodiments, the pharmaceutical composition is formulated for immediate release.

As used herein, the term “delayed release” includes a pharmaceutical preparation, e.g., an orally administered formulation, which passes through the stomach substantially intact and dissolves in the small and/or large intestine (e.g., the colon). In some embodiments, delayed release of the active agent (e.g., a therapeutic as described herein) results from the use of an enteric coating of an oral medication (e.g., an oral dosage form).

The term an “effective amount” of an agent, as used herein, is that amount sufficient to effect beneficial or desired results, such as clinical results, and, as such, an “effective amount” depends upon the context in which it is being applied.

The terms “extended release” or “sustained release” interchangeably include a drug formulation that provides for gradual release of a drug over an extended period of time, e.g., 6-12 hours or more, compared to an immediate release formulation of the same drug. Preferably, although not necessarily, results in substantially constant blood levels of a drug over an extended time period that are within therapeutic levels and fall within a peak plasma concentration range that is between, for example, 0.05-10 μM, 0.1-10 μM, 0.1-5.0 μM, or 0.1-1 μM.

As used herein, the terms “formulated for enteric release” and “enteric formulation” include pharmaceutical compositions, e.g., oral dosage forms, for oral administration able to provide protection from dissolution in the high acid (low pH) environment of the stomach. Enteric formulations can be obtained by, for example, incorporating into the pharmaceutical composition a polymer resistant to dissolution in gastric juices. In some embodiments, the polymers have an optimum pH for dissolution in the range of approx. 5.0 to 7.0 (“pH sensitive polymers”). Exemplary polymers include methacrylate acid copolymers that are known by the trade name Eudragit® (e.g., Eudragit® L100, Eudragit® S100, Eudragit® L-30D, Eudragit® FS 30D, and Eudragit® L100-55), cellulose acetate phthalate, cellulose acetate trimellitiate, polyvinyl acetate phthalate (e.g., Coaterie), hydroxyethylcellulose phthalate, hydroxypropyl methylcellulose phthalate, or shellac, or an aqueous dispersion thereof. Aqueous dispersions of these polymers include dispersions of cellulose acetate phthalate (Aquateric®) or shellac (e.g., MarCoat 125 and 125N). An enteric formulation reduces the percentage of the administered dose released into the stomach by at least 50%, 60%, 70%, 80%, 90%, 95%, or even 98% in comparison to an immediate release formulation. Where such a polymer coats a tablet or capsule, this coat is also referred to as an “enteric coating.”

The term “immediate release” includes where the agent (e.g., therapeutic), as formulated in a unit dosage form, has a dissolution release profile under in vitro conditions in which at least 55%, 65%, 75%, 85%, or 95% of the agent is released within the first two hours of administration to, e.g., a human. Desirably, the agent formulated in a unit dosage has a dissolution release profile under in vitro conditions in which at least 50%, 65%, 75%, 85%, 90%, or 95% of the agent is released within the first 30 minutes, 45 minutes, or 60 minutes of administration.

The term “pharmaceutical composition,” as used herein, includes a composition containing a compound described herein (e.g., an extract of Artemisia dracunculus (Russian Tarragon) leaves, an extract of Melia azedarach (Chinaberry) leaves, an extract of Saururus cernuus (Lizard's Tail) Roots, and an extract of Sambucus Canadensis (Elderberry) leaves, or any pharmaceutically acceptable salt, solvate, or prodrug thereof), formulated with a pharmaceutically acceptable excipient, and typically manufactured or sold with the approval of a governmental regulatory agency as part of a therapeutic regimen for the treatment of disease in a mammal.

Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

A “pharmaceutically acceptable excipient,” as used herein, includes any ingredient other than the compounds described herein (for example, a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, cross-linked polyvinyl pyrrolidone, citric acid, crospovidone, cysteine, ethylcellulose, gelatin, hydroxypropyl cellulose, hydroxypropyl methylcellulose, lactose, magnesium stearate, maltitol, maltose, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable prodrugs” as used herein, includes those prodrugs of the compounds of the present invention which are, within the scope of sound medical judgment, suitable for use in contact with the tissues of humans and animals with undue toxicity, irritation, allergic response, and the like, commensurate with a reasonable benefit/risk ratio, and effective for their intended use, as well as the zwitterionic forms, where possible, of the compounds of the invention.

The term “pharmaceutically acceptable salt,” as use herein, includes those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds of the invention or separately by reacting the free base group with a suitable organic or inorganic acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The terms “pharmaceutically acceptable solvate” or “solvate,” as used herein, includes a compound of the invention wherein molecules of a suitable solvent are incorporated in the crystal lattice. A suitable solvent is physiologically tolerable at the administered dose. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

The term “prevent,” as used herein, includes prophylactic treatment or treatment that prevents one or more symptoms or conditions of a disease, disorder, or conditions described herein (e.g., a foam cell associated diseases). Treatment can be initiated, for example, prior to (“pre-exposure prophylaxis”) or following (“post-exposure prophylaxis”) an event that precedes the onset of the disease, disorder, or conditions. Treatment that includes administration of a compound of the invention, or a pharmaceutical composition thereof, can be acute, short-term, or chronic. The doses administered may be varied during the course of preventive treatment.

The term “prodrug,” as used herein, includes compounds which are rapidly transformed in vivo to the parent compound of the above formula. Prodrugs also encompass bioequivalent compounds that, when administered to a human, lead to the in vivo formation of therapeutic. A thorough discussion is provided in T. Higuchi and V. Stella, Pro-drugs as Novel Delivery Systems, Vol. 14 of the A.C.S. Symposium Series, and Edward B. Roche, ed., Bioreversible Carriers in Drug Design, American Pharmaceutical Association and Pergamon Press, 1987, each of which is incorporated herein by reference. Preferably, prodrugs of the compounds of the present invention are pharmaceutically acceptable.

As used herein, and as well understood in the art, “treatment” includes an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e. not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Treatment” can also mean prolonging survival as compared to expected survival if not receiving treatment. As used herein, the terms “treating” and “treatment” can also include delaying the onset of, impeding or reversing the progress of, or alleviating either the disease or condition to which the term applies, or one or more symptoms of such disease or condition.

The term “unit dosage forms” includes physically discrete units suitable as unitary dosages for human subjects and other mammals, each unit containing a predetermined quantity of active material calculated to produce the desired therapeutic effect, in association with any suitable pharmaceutical excipient or excipients.

As used herein, the term “plasma concentration” includes the amount of therapeutic present in the plasma of a treated subject (e.g., as measured in a rabbit using an assay described below or in a human).

Various objects, features, aspects, and advantages of the present invention will become more apparent from the following detailed description of preferred embodiments of the invention, along with the accompanying drawings in which like numerals represent like components. The present invention may address one or more of the problems and deficiencies of the current technology discussed above. However, it is contemplated that the invention may prove useful in addressing other problems and deficiencies in a number of technical areas. Therefore the claimed invention should not necessarily be construed as limited to addressing any of the particular problems or deficiencies discussed herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are incorporated in and constitute a part of the specification, illustrate various embodiments of the invention and together with the general description of the invention given above and the detailed description of the drawings given below, serve to explain the principles of the invention. It is to be appreciated that the accompanying drawings are not necessarily to scale since the emphasis is instead placed on illustrating the principles of the invention. The invention will now be described, by way of example, with reference to the accompanying drawings in which:

FIG. 1 is a graph showing ethanol extracts of botanicals do not decrease macrophage viability. RAW264.7 macrophages were treated with botanical extracts at 25 μg/mL or with DMSO. Macrophage viability was measured at the indicated timepoints using CellTiter®-Blue assay.

FIGS. 2A and 2B are six photographs and three graphs showing select extracts reduce oxLDL-elicited foam cell formation. RAW264.7 macrophages were pre-treated with botanical extracts (25 μg/mL) or DMSO (0.25%) for one hour followed by incubation with 10 μg/mL oxLDL for 24 hours. Immunofluorescent confocal microscopy was performed following Nile Red staining (2A), and neutral lipid inclusions were quantified using a high content imaging platform (2B). Data represents the average±SEM of three independent experiments. * indicates statistical difference compared to oxLDL DMSO group (p<0.05). CL=Chinaberry Leaves extract; RT=Russian Tarragon extract; LT=Lizard's Tail extract; ELD=Elderberry extract.

FIGS. 3A and 3B are two graphs showing Russian Tarragon extract reduces oxLDL internalization. (3A) RAW264.7 macrophages were pre-treated with botanical extracts (25 μg/mL) or DMSO (0.25%) for one hour, then left untreated or treated with 10 μg/mL oxLDL for 24 hours. Surface expression of CD36 was determined by flow cytometry. (3B) RAW264.7 macrophages were pre-treated with botanical extracts (25 μg/mL) or DMSO (0.25%) for one hour followed by incubation with fluorescently labeled DiI-oxLDL for 3 hours. Internalization of DiI-oxLDL was determined by flow cytometry. Data is expressed as the average of the mean fluorescent intensity (MFI)±SEM of three independent experiments. * Indicates statistical difference compared to oxLDL DMSO group (p<0.05).

FIGS. 4A-4D are two graphs and two Western blot analyses showing Russian Tarragon extract reverses foam cell intracellular lipid inclusions. 4A) RAW 264.7 macrophages were loaded with 10 μg/ml oxLDL for 24 hours before being treated with 25 μg/ml botanical extracts. 24 hours alter foam cell formation was quantified using Cellomcs (n=3). 4B) RAW 264.7 macrophages were loaded with 10 μg/ml oxLDL containing ³H Cholesterol for 24 hours. Cells were then treated with 25 μg/ml botanical extracts for 24 hours. Supernatant and Cells were collected and radioactivity was determined. Efflux was determined as supernatant count/(supernantant+Cell count)*100 (n=3). 4C) RAW 264.7 macrophages were treated with 25 μg/ml China berry extracts and then stimulated with 10 μg/ml oxLDL for twenty four hours in presence of lysosomal inhibitor. Protein was isolated and LC3II levels were determined by Western blot analysis (representative blot of three independent experiments). 4D) RAW 264.7 macrophages were loaded with 10 μg/ml for 24 hours. Cells were then treated with 25 μg/ml Russian Tarragon extracts hours in presence of lysosomal inhibitor. Protein was isolated 4 and 8 hours after addition of RT and LC3II levels were determined by Western blot analysis (representative blot of three independent experiments).

FIGS. 5A and 5B are two graphs showing select ethanol extracts reduce foam cell IL-6 production. RAW264.7 macrophages were pre-treated with botanical extracts (25 μg/mL) or DMSO (0.25%) for one hour followed by incubation with 10 μg/mL oxLDL for 48 hours. IL-6 in supernatants was quantified by ELISA. *Indicates statistical difference compared to oxLDL DMSO group (p<0.05).

### DETAILED DESCRIPTION OF THE INVENTION

The present invention will be understood by reference to the following detailed description, which should be read in conjunction with the appended drawings. It is to be appreciated that the following detailed description of various embodiments is by way of example only and is not meant to limit, in any way, the scope of the present invention. In the summary above, in the following detailed description, in the claims below, and in the accompanying drawings, reference is made to particular features (including method steps) of the present invention. It is to be understood that the disclosure of the invention in this specification includes all possible combinations of such particular features, not just those explicitly described. For example, where a particular feature is disclosed in the context of a particular aspect or embodiment of the invention or a particular claim, that feature can also be used, to the extent possible, in combination with and/or in the context of other particular aspects and embodiments of the invention, and in the invention generally. The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm, and whose upper limit is 100 mm. The embodiments set forth the below represent the necessary information to enable those skilled in the art to practice the invention and illustrate the best mode of practicing the invention. In addition, the invention does not require that all the advantageous features and all the advantages need to be incorporated into every embodiment of the invention.

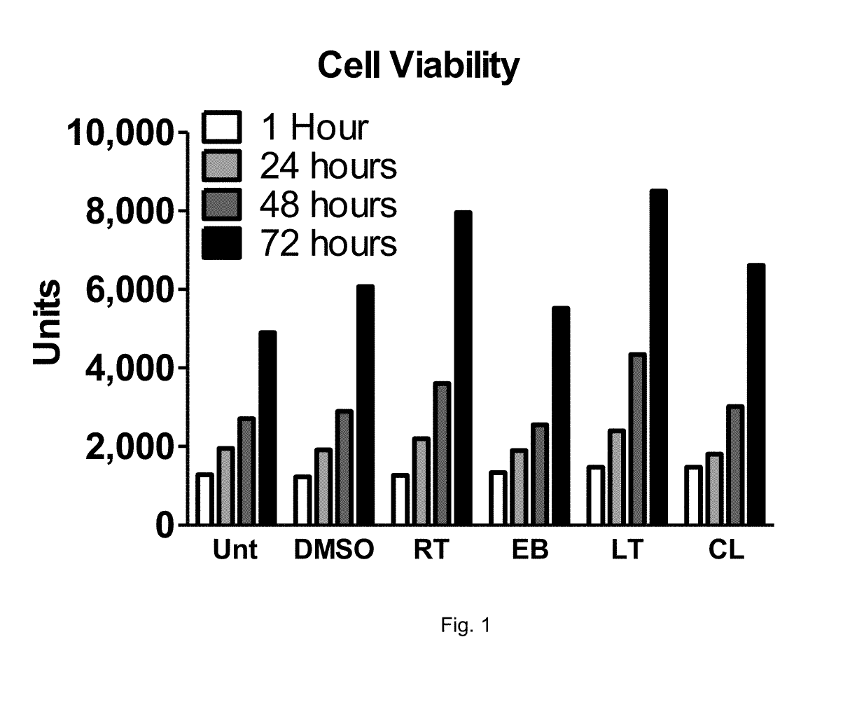

Turning now to FIGS. 1-5B, a brief description concerning the various components of the present invention will now be briefly discussed.

Selected Botanical Extracts do No Inhibit RAW264.7 Cellular Growth.

The inventors first wanted to ensure that the plant extracts did not cause unintended cell death in the inventors' RAW 264.7 macrophage cell line. In an effort to minimize presence of the vehicle (DMSO), the inventors tested extract concentrations <25 μg/ml. The inventors performed a CellTiter-Blue cell viability assay on RAW264.7 macrophage treated with 5-25 μg/ml of botanical extracts or 0.25% DMSO as the vehicle control. The inventors observed no difference between untreated and vehicle treatment with any analysis performed in the disclosed experiments, thus only vehicle controls are presented in graphs. None of the botanical extracts demonstrated any cytotoxcity towards the inventors' RAW264.7 macrophage cells (FIG. 1) with the exception of chinaberry stems, which caused significant cell death at all concentrations tested (data not shown) and was removed from further study. The extracts of the plants are generally referred to in this description as just the name of the plants. For example, Russian Tarragon extract is referred to simply as Russian Tarragon.

Foam Cell Formation is Inhibited by Select Botanical Extracts.

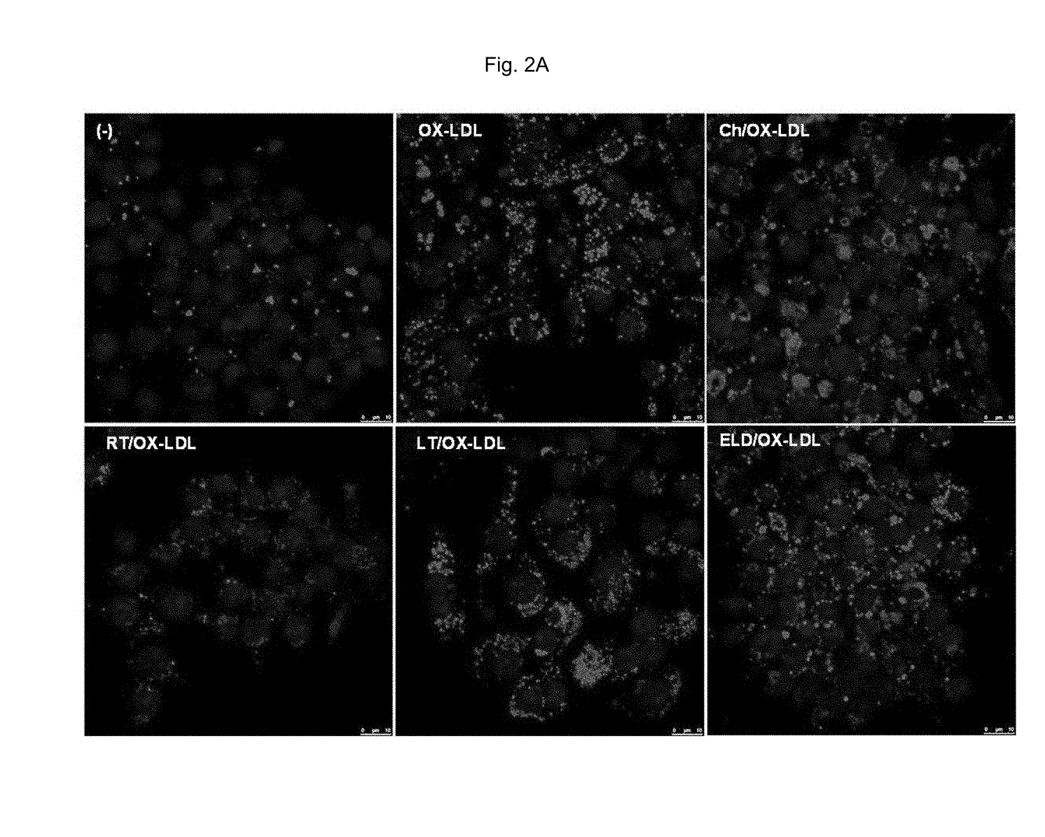

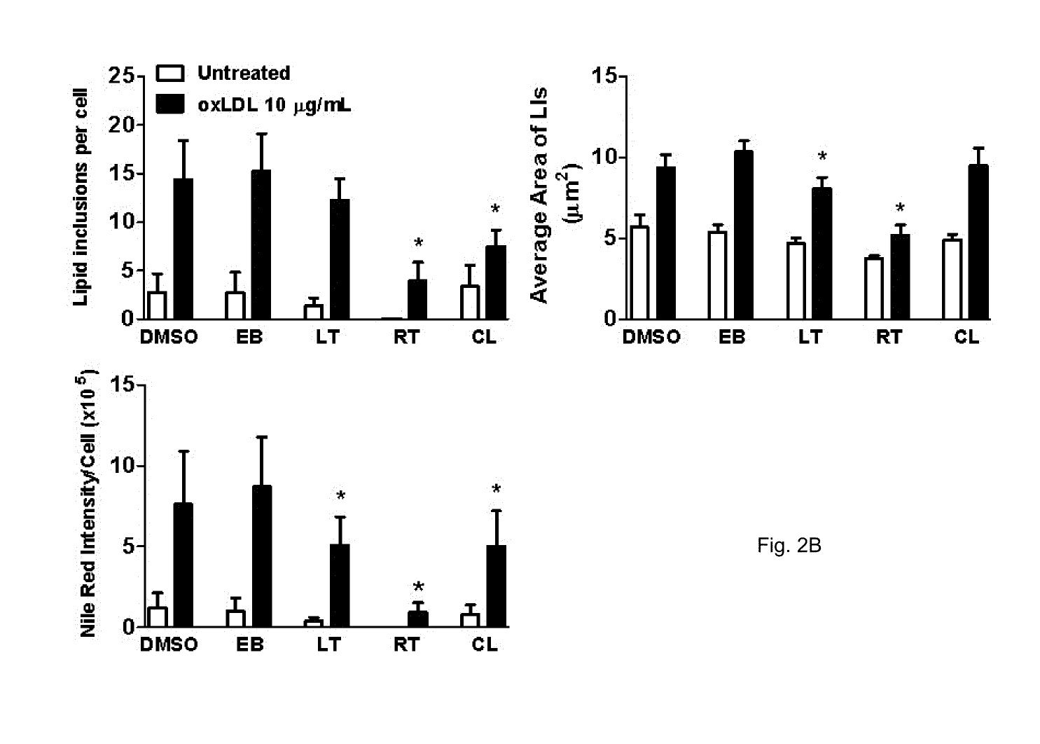

The treatment of macrophages in vitro with modified-LDL is known to generate lipid-laden macrophages that are comparable to plaque macrophage foam cells. To quantify changes in foam cell formation, the inventors used Nile Red, a dye that fluoresces intensely in neutral lipid-rich environments including lipid droplets and cholesterol ester-rich lipoproteins. Initially, confocal fluorescence microscopy was used to visualize changes to Nile red staining in RAW cells oxLDL loaded for 24 hours in the presence or absence of the indicated botanical extracts. FIG. 2A clearly demonstrates that oxLDL treatment results in a lipid laden phenotype compared to untreated RAW cells which exhibit few minute Nile staining structures. This lipid laden phenotype was prevented most completely by Russian Tarragon. Automated epifluorescene microscopy coupled with analysis software (Cellomics) was used to quantify the number, size and staining intensity of intracellular lipid inclusions formed following botanical and oxLDL treatment. Russian Tarragon was the only extract to reduce foam cell formation in all aspects measured (FIG. 2B). Pre-treatment of macrophages with 25 μg/mL Russian Tarragon significantly reduced the number and size of lipid inclusions, as well as overall Nile Red staining intensity. Extracts of Elderberry leaves had no effect on foam cell formation (FIG. 2B). Extracts of both China Berry leaves and Lizard's Tail roots altered at least one aspect of foam cell formation. Lizard's Tail root extract reduced the size of lipid inclusions while Chinaberry leave extract also reduced the number of lipid inclusions within oxLDL stimulated macrophages. Thus, three of the four botanical extracts were able to reduce foam cell formation.

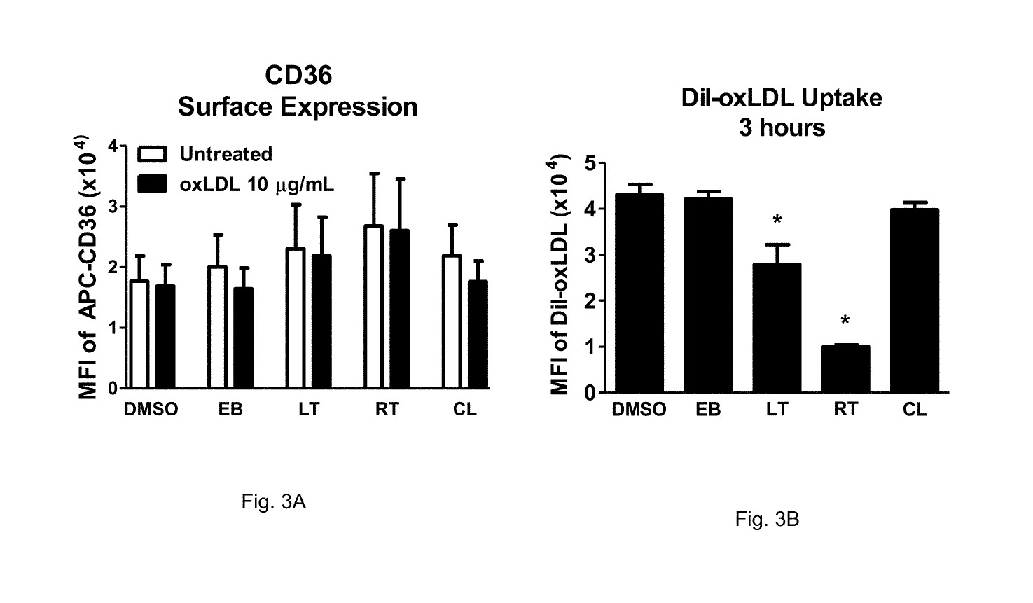

Modified-LDL elicited foam cell formation requires internalization of LDL particles, thus the inventors wished to determine if the inhibition of foam cell formation by extracts was due to a defect in oxLDL uptake. The majority of highly oxidized-LDL is internalized by macrophages through receptor mediated endocytosis via the class B scavenger receptor CD36; as such, the inventors examined the amount of CD36 present at the macrophage membrane following treatment with the extracts. None of the botanical extracts reduced surface expression of CD36 in untreated or oxLDL treated macrophages (FIG. 3A). The inventors next examined the internalization of oxLDL using fluorescently labeled DiI-oxLDL. The inventors measured fluorescent intensity of macrophages three hours following DiI-oxLDL treatment by flow cytometry in the presence or absence of extracts. Both Lizard's Tail root and Russian Tarragon extracts significantly reduced DiI-oxLDL uptake (FIG. 3B). To ensure the inventors were measuring uptake and not just DiI-oxLDL bound to the outer membrane the inventors performed these studies at 4° C., which would allow binding events to occur but not internalization via endocytosis. There was no difference in any treatment groups in the amount of fluorescence in samples incubated at 4° C. (data not shown).

Foam Cell Phenotypes are Reversed by Select Botanical Treatments.

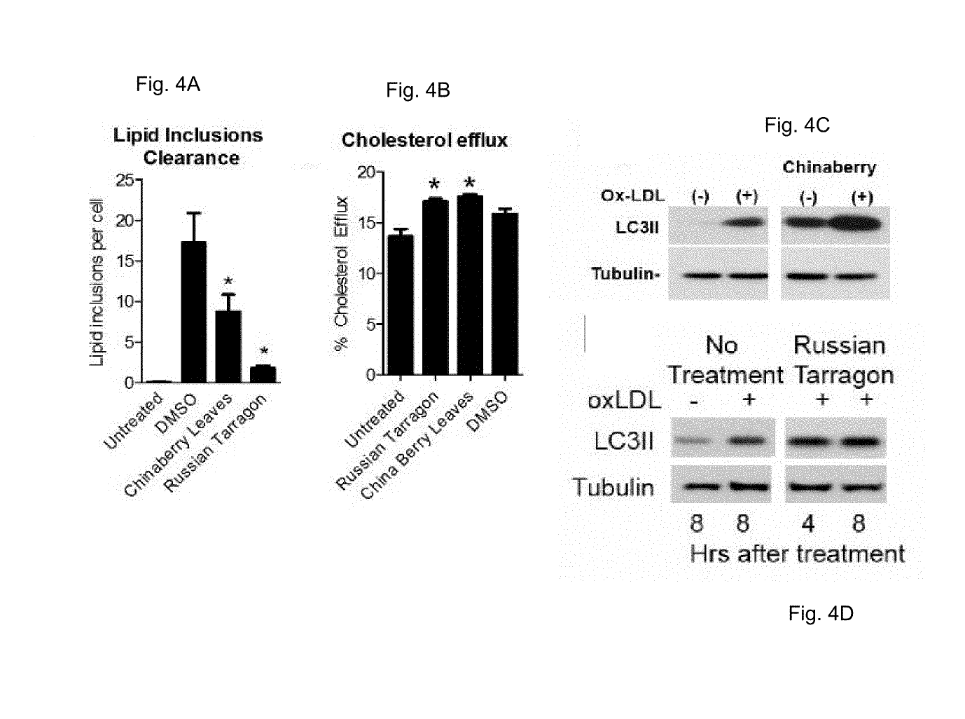

Generation of foam cells begins during fatty streak formation, an event that happens during adolescence. Thus to prevent atherosclerosis, foam cells should preferably be regressed by enhancing cholesterol efflux. The inventors examined if either Russian Tarragon or China Berry Extracts could regress foam cell formation. Macrophages were preloaded with oxLDL for 24 hours then treated with botanical extracts for another 24 hours. Foam cell phenotype was determined by the INLET Cellomics high content imaging platform. The inventors were excited to observe that both Russian Tarragon and Chinaberry leaves reduced lipid inclusions (FIG. 4A). As mentioned above, foam cell regression requires cholesterol efflux in which autophagy can contribute, thus the inventors examined if these extracts enhanced either processes. The inventors performed an HDL cholesterol efflux assay in which macrophages were loaded with radiolabeld cholesterol for 24 hours. The inventors then treated foam cells with botanical extracts and monitored efflux to HDL for 24 hours. Botanical extract treatment enhanced cholesterol efflux to HDL (FIG. 4B). At this time, the inventors also isolated protein to monitor the levels of the autophagic marker LC3II by Western blot analysis. Increases in LC3II in the presence of lysosomal enzyme inhibitor suggest increase autophagy. The inventors observed an increase in LC3II with Chinaberry extracts but not Russian Tarragon (FIG. 4C and data not shown). The lack of increase in LC3II in Russian Tarragon treatment macrophages at 24 hours after treatment may have been due to the fact that by 24 hours after treatment with Russian Tarragon, foam cells formation is almost completely reversed (FIG. 4A), thus the inventors looked at earlier time points. Treatment of loaded foam cells with Russian Tarragon for 4 or 8 hours resulted in increased LC3II levels (FIG. 4D). While acknowledging that proving “prevention” is analogous to proving a negative, these results provide strong evidence that these botanical extracts are able to prevent the development of atherosclerosis by enhancing reverse cholesterol efflux in vivo. That is to say, in a situation where atherosclerosis would likely occur because, inter alia, insufficient cholesterol efflux, these results evidence that these botanical extracts could prevent the atherosclerosis. Stated in another way, it appears that treatment with these botanical extracts treats a pre-atherosclerosis state, preventing it from progressing to atherosclerosis, thereby preventing atherosclerosis. Further, these results also evidence that the rate of fusion of autophagosomes and lysosomes is increased by treatment with Russian Tarragon and Chinaberry extracts.

Foam Cell Inflammatory Responses are Inhibited by Russian Tarragon Extracts.

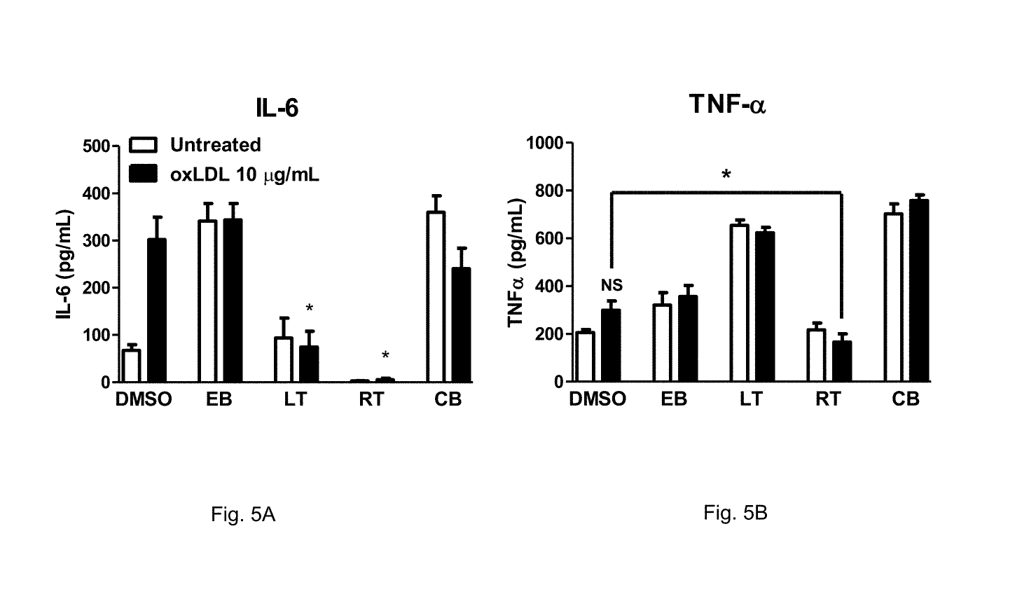

Another key aspect of foam cell biology is the production of inflammatory factors by foam cells. Tumor necrosis factor alpha (TNF-α) and interleukin 6 (IL-6) are cytokines, produced by foam cells, that significantly contribute to the progression of atherosclerosis and the catastrophic events associated with cardiovascular disease. The inventors tested the plant extracts for their ability to reduce oxLDL-elicited TNF-α and IL-6 production by macrophages. The inventors demonstrated that oxLDL-elicited production of TNF-α and IL-6 by RAW264.7 macrophages requires an extended (48 hours) period of incubation. Macrophages were left untreated or incubated in the presence of botanical extracts and oxLDL for 48 hours. Only Russian Tarragon Extract reduced both TNF-α and IL-6 production by oxLDL treated macrophages (FIG. 5). Lizard's Tail root extract significantly reduced oxLDL-elicited IL-6 while Elderberry leaves partially, but not significantly reduced oxLDL-elicited TNF-α. However, Lizard's tail root and Chinaberry leaves extract alone induced TNF-α and Elderberry leaf and Chinaberry leaf extracts induced IL-6 production (FIG. 5). These results suggest that the ethanol extract of Russian Tarragon significantly attenuates oxLDL-elicited inflammatory activity.

Discussion

Atherosclerosis and CVD are conditions that progress for long periods of time before a critical event occurs. The macrophage foam cell formation is known to critically contribute to—as a necessary step in the development of—atherosclerosis and CVD progression from an early point in the conditions, and as such the modification of foam cell biology is a promising candidate for targeted therapeutic intervention as it pertains to atherosclerosis and CVD. Herein, the inventors demonstrate that ethanolic extracts of select plants native to southwest Louisiana inhibit macrophage foam cell formation and inflammatory activity and are evidenced to be therapeutic to and preventative for atherosclerosis and CVD conditions.

Stimulation of macrophages with oxLDL results in foam cell formation, through both unregulated uptake of oxLDL and reduce efflux of cholesterol. The inventors used this model of foam cell formation to investigate if botanical extracts could inhibit foam cell formation. The ethanol extracts of Russian Tarragon, Lizard's Tail root and Chinaberry leaf all inhibited oxLDL-elicited foam cell formation (accumulation of intracellular lipid inclusions), to varying degrees. Chinaberry leaf extract significantly reduced the number, and to a lesser extent, the staining intensity of intracellular lipid inclusions, while not altering their size. RT extract was most effective at blocking macrophage foam cell formation, as it reduced the average size, number and staining intensity of intracellular lipid inclusions. The inventors wanted to determine if this reduction in foam cell formation was due to inhibition of oxLDL uptake. Scavenger receptors are responsible for recognizing and initiating endocytosis of modified LDLs. Though there are numerous scavenger receptors, CD36 is responsible for recognition and endocytosis of oxLDL. Reduction in CD36 levels will reduce oxLDL uptake. None of the botanical extracts reduce CD36 surface levels. To monitor endocytosis of oxLDL, the inventors tracked the engulfment of fluorescently labeled oxLDL. Of the three extracts that blocked foam cell formation only Russian Tarragon inhibited oxLDL internalization.

Because patients presenting with MetS and or atherosclerosis likely already have a significant accumulation of foam cells, a desirable trait in a therapeutic would be the reversal of the macrophage lipid-laden phenotype. To this end, the inventors investigated the ability of the botanical extracts to reduce the foam cell phenotype of oxLDL loaded macrophages. Extracts of both Russian Tarragon and Chinaberry leaves significantly reduced the number of lipid inclusions in foam cells. It is possible extracts that lead to the clearance of lipid inclusions are stimulating enhanced cholesterol efflux. Increasing the rate of cholesterol efflux from macrophage requires either an increase in the expression of cholesterol transporters such as ABCA1, increased activity of the lysosomal acid lipase enzyme, and/or augmentation of autophagic flux. The inventors observed that these extracts do not increase the expression of cholesterol transporter mRNA, but rather they increase the rate of autophagy (Data not shown). These results highlight that extracts of Russian Tarragon and Chinaberry leaves are able to alter lipid and/or cholesterol handling by macrophages to both prevent and reduce foam cell formation, thus treating a condition of MetS and atherosclerosis.

Inflammatory cytokine production is another key aspect of foam cell biology. The inventors hypothesized that these extracts would block oxLDL-elicited pro-inflammatory cytokine production. There is conflicting data as to how oxLDL elicited foam cell formation. The inventors' previous work and the work of others have demonstrated that treatment of macrophages with oxLDL initially inhibits pro-inflammatory responses (within the first 24 hours). However, the inventors demonstrated that chronic exposure (48 hours) of macrophages with oxLDL eventually results in production of pro-inflammatory mediators IL-6 and TNFα. Treatment of macrophages with extracts of EB, LT and CB all displayed some induction of IL-6 or TNFα in the absence of oxLDL treatment. In contrast, Russian tarragon significantly inhibited elicitation of both IL-6 and TNFα. As such, Russian Tarragon extracts were capable of inhibiting both foam cell formation and pro-inflammatory response, as well as inducing foam cell regression. Inflammation or chronic inflammation is known or suggested to be associated with many diseases, including asthma, arthritis, Crohn's disease, Alzheimer's disease, cancer, cardiovascular disease, diabetes, high blood pressure, high cholesterol levels and Parkinson's disease, for example. The inventors' experiments evidence that RT extract is an effective treatment of such diseases or of pre-diseases states of such diseases. Future work will more definitely define the mechanism by which the different botanical extracts modulate foam cell biology. The disclosed work evidences that the botanical extracts are a source of compounds that can treat inflammatory conditions mediated by macrophages including MetS and cardiovascular disease.

Two polyphenolic compounds that impact glucose blood levels were isolated from Russian Tarragon and identified as 6-demethoxycapillarisin and 2_,4_-dihydroxy-4-methoxydihydrochalcone. These compounds have not been tested to see if they impact macrophage foam cell formation, but are candidates for such test. The inventors also will investigate if Russian Tarragon and China Berry combined together act synergistically. Finally a number of flavonoids and other polyphenols, including EGCG, luteolin, ellagic acid, quercetin to name a few, have been suggested to alter one aspect or another of foam cell biology, suggesting they also might act synergistically with Russian Tarragon and/or Chinaberry Extracts. Further, the reduction and treatment of foam cell formation would additionally treat other foam cell related diseases, including kidney disease and those where lipid droplet formation and autophagic flux play a role include neurological disorders (Parkinson's), cancers and tuberculosis. The effective dosages of therapeutic per patient mass would are preferably between 50 to 2000 mg/kg, more preferably between 200 and 1000 mg/kg, and most preferably at 500 mg/kg.

Materials and Methods. Preparation of Plant Extracts.

Plants were lyophilized and extracted in 80% ethanol 10:1 by sonication in a 50° C. water bath for 1 h. Extracts were filtered through Miracloth (Calbiochem, Billerica, Mass., USA) and centrifuged to obtain a clear solution. The clear extracts were then dried by rotary evaporation followed by lyophilization and stored at −20° C. The extracts were resuspended in dimethyl sulfoxide or ethanol and diluted to the indicated concentrations in cell culture media for use in the screening assays.

Cell Lines.

The murine macrophage cell line RAW264.7 (ATCC #TIB-71) was cultured at 37° C., 5% CO₂ in DMEM supplemented with 10% FBS (ATLAS), 2 mM L-glutamine, 1 mM sodium pyruvate (HyClone), 100 U/mL penicillin, 100 μg/mL streptomycin (ATCC).

Preparation of Oxidized LDLs.

Human LDLs (Intracel or Kalen Biomedical) were oxidized by incubation with copper (II) sulfate (CuSO₄) as previously described by Yurdagul A, Jr., Green J, Albert P, McInnis M C, Mazar A P, Orr A W. alpha5beta1 integrin signaling mediates oxidized low-density lipoprotein-induced inflammation and early atherosclerosis. Arterioscler Thromb Vasc Biol. 2014; 34:1362-73. Briefly, human LDL was dialyzed against phosphate buffered saline (PBS) pH 7.6 for 24 hours at room temperature using a 7,000 MWCO Slide-A-Lyzer® Dialysis cassette (Thermo Scientific) to remove EDTA. The buffer was then changed to fresh PBS containing 13.8 μM CuSO₄. The LDL was incubated with CuSO₄ for 72 hours at room temperature. The buffer was then changed to fresh PBS containing 50 μM EDTA for 24 hours at 4° C. to remove excess copper. The buffer was then changed to fresh PBS containing 50 μM EDTA and allowed to incubate an additional 24 hours. Oxidized LDL (oxLDL) was then collected and stored under argon gas at 4° C. It has been described that this methodology results in oxLDL that consistently displays a relative electrophoretic mobility between 2 and 3. All steps performed under sterile conditions.

Enzyme Immunoassays.

TNF-α and IL-6 (eBioscience) were measured using commercially available immunoassay kits according to manufacturer's instructions.

Macrophage Imaging and Quantification of Intracellular Lipid Content.

5,000-10,000 RAW264.7 macrophages were seeded into a sterile, black-walled 96-well half-area high content imaging plate with a 0.2 mm glass bottom (Corning 4580) and incubated overnight at 37° C. to allow cells to adhere. For experiments to inhibit foam cell formation, the macrophages were mock treated (PBS), treated with 25 μg/ml of botanical extract one hour prior to being stimulated with 10 μg/mL oxLDL for 24 hours at 37° C. For experiments investigating reversion of the foam cell phenotype macrophages were treated with 10 μg/mL oxLDL for 24 hours at 37° C. then treated with 25 μg/ml of botanical extract and 10 μg/mL oxLDL for another 24 hours at 37° C. The media was then aspirated and the cells were washed once with PBS. The macrophages were then fixed with 4% formaldehyde in PBS for 20 minutes at room temperature. Following fixation, the macrophages were washed once with PBS and then incubated with 100 nM Nile Red (ACROS Organics) in PBS for 5 minutes at room temperature protected from light. Following Nile Red incubation, the macrophages were washed once with PBS and incubated with Hoechst solution (500 nM in PBS) for 30 minutes at room temperature protected from light. The Hoechst solution was then removed and PBS added to each well. The plates were scanned using the Cellomics imaging system (Thermo Scientific). Lipid inclusion images presented as an inverted grayscale image with densely stained areas in black. Approximately 3,000 cells were analyzed per treatment group per experiment for quantitation of macrophage images.

Confocal Microscopy.

Cells were grown to 60% confluency on glass coverslips in a 6-well plate. Following treatments cells were fixed in 4% PFA and washed twice with PBS. Fixed cells were stained with 100 nM Nile Red and DAPI in PBS for 5 minutes. Coverslips were washed again three times with PBS and then mounted on glass slides with SlowFade Gold antifade reagent (Invitrogen). Representative images were taken on a Leica TCS SP5 Confocal Microscope at 60× magnification with oil immersion. Images are shown as equivalently enhanced using ImageJ software.

Flow Cytometry.

CD36 surface expression: Macrophages were mock treated (PBS), treated with 25 μg/ml of botanical extract one hour prior to being stimulated with 10 μg/mL oxLDL for 24 hours at 37° C. Macrophages were washed once with FACS wash (1% BSA, 0.1% sodium azide in PBS) and re-suspended in 400 μL FACS wash. Macrophages were treated with anti-mouse CD16/32 (1:400) in FACS wash for 30 minutes on ice to block Fc Receptors and then incubated with anti-mouse CD36 antibody (APC) (1:400) for 30 minutes on ice. The macrophages were fixed (4% formaldehyde in PBS) for 20 minutes on ice. Cells were stored at 4° C. protected from light until analysis.

For uptake of oxLDL, macrophages were mock treated (PBS), treated with oxLDL (10 μg/mL) or with 1:9 Dil-oxLDL/unlabled oxLDL at 37° C. The cells were then fixed, stored and analyzed by flow cytometry as above.

Viability Assay.

RAW 264.7 murine macrophage cells were plated at a density of 5000 cells per well in clear 96-well plates in 50 μL of complete DMEM (10% FBS and 1% pen-strep). The following day, compounds were added to the plates in the appropriate wells at a 2× concentration in 50 μL DMEM. Once compounds had been added, 20 μL of CellTiter®-Blue reagent (Promega) was added to each well of the 0 time point plate. Plates were incubated for 1 hour at 37° C. and 5% CO₂ before fluorescence was measured at 560_{Ex}/590_{Em} on a Synergy 4 plate reader (BioTek). CellTiter®-Blue measures the reduction of an indicator dye resazurin to the fluorescent product resorufin in live cells. Fluorescence was measured on the remaining plates at 24, 48, and 72 hour time points and graphs were generated.

Statistical Analysis.

GraphPad Prism 5.0 was used for statistical analyses. Statistical significance was determined using a one-way ANOVA analysis of variance with a Dunnett's post-test, P≦0.05.

Pharmaceutical Compositions

The methods described herein can also include the administrations of pharmaceutically acceptable compositions that include the therapeutic, or a pharmaceutically acceptable salt, solvate, or prodrug thereof. When employed as pharmaceuticals, any of the present compounds can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical, parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, by suppositories, or oral administration.

This invention also includes pharmaceutical compositions which can contain one or more pharmaceutically acceptable carriers. In making the pharmaceutical compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, powders, lozenges, sachets, cachets, elixirs, suspensions, emulsions, solutions, syrups, and soft and hard gelatin capsules. As is known in the art, the type of diluent can vary depending upon the intended route of administration. The resulting compositions can include additional agents, such as preservatives.

The therapeutic agents of the invention can be administered alone, or in a mixture, in the presence of a pharmaceutically acceptable excipient or carrier. The excipient or carrier is selected on the basis of the mode and route of administration. Suitable pharmaceutical carriers, as well as pharmaceutical necessities for use in pharmaceutical formulations, are described in Remington: The Science and Practice of Pharmacy, 22^{nd} Ed., Gennaro, Ed., Lippencott Williams & Wilkins (2012), a well-known reference text in this field, and in the USP/NF (United States Pharmacopeia and the National Formulary), each of which is incorporated by reference. In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

Examples of suitable excipients are lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. Other exemplary excipients are described in Handbook of Pharmaceutical Excipients, 8^{th} Edition, Sheskey et al., Eds., Pharmaceutical Press (2017), which is incorporated by reference.

The methods described herein can include the administration of a therapeutic, or prodrugs or pharmaceutical compositions thereof, or other therapeutic agents. Exemplary therapeutics include those that alter lipid and/or cholesterol handling by macrophages to both prevent and reduce foam cell formation and/or reduced one or both of TNF-α and IL-6 production by macrophages.

The pharmaceutical compositions can be formulated so as to provide immediate, extended, or delayed release of the active ingredient after administration to the patient by employing procedures known in the art.

The compositions can be formulated in a unit dosage form, each dosage containing, e.g., 0.1-500 mg of the active ingredient. For example, the dosages can contain from about 0.1 mg to about 50 mg, from about 0.1 mg to about 40 mg, from about 0.1 mg to about 20 mg, from about 0.1 mg to about 10 mg, from about 0.2 mg to about 20 mg, from about 0.3 mg to about 15 mg, from about 0.4 mg to about 10 mg, from about 0.5 mg to about 1 mg; from about 0.5 mg to about 100 mg, from about 0.5 mg to about 50 mg, from about 0.5 mg to about 30 mg, from about 0.5 mg to about 20 mg, from about 0.5 mg to about 10 mg, from about 0.5 mg to about 5 mg; from about 1 mg from to about 50 mg, from about 1 mg to about 30 mg, from about 1 mg to about 20 mg, from about 1 mg to about 10 mg, from about 1 mg to about 5 mg; from about 5 mg to about 50 mg, from about 5 mg to about 20 mg, from about 5 mg to about 10 mg; from about 10 mg to about 100 mg, from about 20 mg to about 200 mg, from about 30 mg to about 150 mg, from about 40 mg to about 100 mg, from about 50 mg to about 100 mg of the active ingredient, from about 50 mg to about 300 mg, from about 50 mg to about 250 mg, from about 100 mg to about 300 mg, or, from about 100 mg to about 250 mg of the active ingredient. For preparing solid compositions such as tablets, the principal active ingredient is mixed with one or more pharmaceutical excipients to form a solid bulk formulation composition containing a homogeneous mixture of a compound of the present invention. When referring to these bulk formulation compositions as homogeneous, the active ingredient is typically dispersed evenly throughout the composition so that the composition can be readily subdivided into equally effective unit dosage forms such as tablets and capsules. This solid bulk formulation is then subdivided into unit dosage forms of the type described above containing from, for example, 0.1 to about 500 mg of the active ingredient of the present invention.

Compositions for Oral Administration

The pharmaceutical compositions contemplated by the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Formulations for oral administration may also be presented as chewable tablets, as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active drug by controlling the dissolution and/or the diffusion of the active drug substance. Any of a number of strategies can be pursued in order to obtain controlled release and the targeted plasma concentration vs time profile. In one example, controlled release is obtained by appropriate selection of various formulation parameters and ingredients, including, e.g., various types of controlled release compositions and coatings. Thus, the drug is formulated with appropriate excipients into a pharmaceutical composition that, upon administration, releases the drug in a controlled manner. Examples include single or multiple unit tablet or capsule compositions, oil solutions, suspensions, emulsions, microcapsules, microspheres, nanoparticles, patches, and liposomes. In certain embodiments, compositions include biodegradable, pH, and/or temperature-sensitive polymer coatings.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions suitable for oral mucosal administration (e.g., buccal or sublingual administration) include tablets, lozenges, and pastilles, where the active ingredient is formulated with a carrier, such as sugar, acacia, tragacanth, or gelatin and glycerine.

Coatings

The pharmaceutical compositions formulated for oral delivery, such as tablets or capsules of the present invention can be coated or otherwise compounded to provide a dosage form affording the advantage of delayed or extended release. The coating may be adapted to release the active drug substance in a predetermined pattern (e.g., in order to achieve a controlled release formulation) or it may be adapted not to release the active drug substance until after passage of the stomach, e.g., by use of an enteric coating (e.g., polymers that are pH-sensitive (“pH controlled release”), polymers with a slow or pH-dependent rate of swelling, dissolution or erosion (“time-controlled release”), polymers that are degraded by enzymes (“enzyme-controlled release” or “biodegradable release”) and polymers that form firm layers that are destroyed by an increase in pressure (“pressure-controlled release”)). Exemplary enteric coatings that can be used in the pharmaceutical compositions described herein include sugar coatings, film coatings (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or coatings based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose. Furthermore, a time delay material such as, for example, glyceryl monostearate or glyceryl distearate, may be employed.

For example, the tablet or capsule can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release.

When an enteric coating is used, desirably, a substantial amount of the drug is released in the lower gastrointestinal tract.

In addition to coatings that effect delayed or extended release, the solid tablet compositions may include a coating adapted to protect the composition from unwanted chemical changes (e.g., chemical degradation prior to the release of the active drug substance). The coating may be applied on the solid dosage form in a similar manner as that described in Encyclopedia of Pharmaceutical Technology, vols. 5 and 6, Eds. Swarbrick and Boyland, 2000.

Parenteral Administration

Within the scope of the present invention are also parenteral depot systems from biodegradable polymers. These systems are injected or implanted into the muscle or subcutaneous tissue and release the incorporated drug over extended periods of time, ranging from several days to several months. Both the characteristics of the polymer and the structure of the device can control the release kinetics which can be either continuous or pulsatile. Polymer-based parenteral depot systems can be classified as implants or microparticles. The former are cylindrical devices injected into the subcutaneous tissue whereas the latter are defined as spherical particles in the range of 10-100 μm. Extrusion, compression or injection molding are used to manufacture implants whereas for microparticles, the phase separation method, the spray-drying technique and the water-in-oil-in-water emulsion techniques are frequently employed. The most commonly used biodegradable polymers to form microparticles are polyesters from lactic and/or glycolic acid, e.g. poly(glycolic acid) and poly(L-lactic acid) (PLG/PLA microspheres). Of particular interest are in situ forming depot systems, such as thermoplastic pastes and gelling systems formed by solidification, by cooling, or due to the sol-gel transition, cross-linking systems and organogels formed by amphiphilic lipids. Examples of thermosensitive polymers used in the aforementioned systems include, N-isopropylacrylamide, poloxamers (ethylene oxide and propylene oxide block copolymers, such as poloxamer 188 and 407), poly(N-vinyl caprolactam), poly(siloethylene glycol), polyphosphazenes derivatives and PLGA-PEG-PLGA.

Mucosal Drug Delivery

Mucosal drug delivery (e.g., drug delivery via the mucosal linings of the nasal, rectal, vaginal, ocular, or oral cavities) can also be used in the methods described herein. Methods for oral mucosal drug delivery include sublingual administration (via mucosal membranes lining the floor of the mouth), buccal administration (via mucosal membranes lining the cheeks), and local delivery (Harris et al., Journal of Pharmaceutical Sciences, 81(1): 1-10, 1992).

Oral transmucosal absorption is generally rapid because of the rich vascular supply to the mucosa and allows for a rapid rise in blood concentrations of the therapeutic.

For buccal administration, the compositions may take the form of, e.g., tablets, lozenges, etc. formulated in a conventional manner. Permeation enhancers can also be used in buccal drug delivery. Exemplary enhancers include 23-lauryl ether, aprotinin, azone, benzalkonium chloride, cetylpyridinium chloride, cetyltrimethylammonium bromide, cyclodextrin, dextran sulfate, lauric acid, lysophosphatidylcholine, methol, methoxysalicylate, methyloleate, oleic acid, phosphatidylcholine, polyoxyethylene, polysorbate 80, sodium EDTA, sodium glycholate, sodium glycodeoxycholate, sodium lauryl sulfate, sodium salicylate, sodium taurocholate, sodium taurodeoxycholate, sulfoxides, and alkyl glycosides. Bioadhesive polymers have extensively been employed in buccal drug delivery systems and include cyanoacrylate, polyacrylic acid, hydroxypropyl methylcellulose, and poly methacrylate polymers, as well as hyaluronic acid and chitosan.

Liquid drug formulations (e.g., suitable for use with nebulizers and liquid spray devices and electrohydrodynamic (EHD) aerosol devices) can also be used. Other methods of formulating liquid drug solutions or suspension suitable for use in aerosol devices are known to those of skill in the art (see, e.g., Biesalski, U.S. Pat. No. 5,112,598, and Biesalski, U.S. Pat. No. 5,556,611).

Formulations for sublingual administration can also be used, including powders and aerosol formulations. Exemplary formulations include rapidly disintegrating tablets and liquid-filled soft gelatin capsules.

Dosing Regimes

The present methods for treating foam cell associated disease are carried out by administering a therapeutic for a time and in an amount sufficient to result in decreased foam cell formation or decreased inflammation.

The amount and frequency of administration of the compositions can vary depending on, for example, what is being administered, the state of the patient, and the manner of administration. In therapeutic applications, compositions can be administered to a patient suffering from foam cell associated disease in an amount sufficient to relieve or least partially relieve the symptoms of the foam cell associated disease and its complications. The dosage is likely to depend on such variables as the type and extent of progression of the foam cell associated disease, the severity of the foam cell associated disease, the age, weight and general condition of the particular patient, the relative biological efficacy of the composition selected, formulation of the excipient, the route of administration, and the judgment of the attending clinician. Effective doses can be extrapolated from dose-response curves derived from in vitro or animal model test system. An effective dose is a dose that produces a desirable clinical outcome by, for example, improving a sign or symptom of the foam cell associated disease or slowing its progression.

The amount of therapeutic per dose can vary. For example, a subject can receive from about 0.1 μg/kg to about 10,000 μg/kg. Generally, the therapeutic is administered in an amount such that the peak plasma concentration ranges from 150 nM-250 μM.

Exemplary dosage amounts can fall between 0.1-5000 μg/kg, 100-1500 μg/kg, 100-350 μg/kg, 340-750 μg/kg, or 750-1000 μg/kg. Exemplary dosages can 0.25, 0.5, 0.75, 1°, or 2 mg/kg. In another embodiment, the administered dosage can range from 0.05-5 mmol of therapeutic (e.g., 0.089-3.9 mmol) or 0.1-50 μmol of therapeutic (e.g., 0.1-25 μmol or 0.4-20 μmol).

The plasma concentration of therapeutic can also be measured according to methods known in the art. Exemplary peak plasma concentrations of therapeutic can range from 0.05-10 μM, 0.1-10 μM, 0.1-5.0 μM, or 0.1-1 μM. Alternatively, the average plasma levels of therapeutic can range from 400-1200 μM (e.g., between 500-1000 μM) or between 50-250 μM (e.g., between 40-200 μM). In some embodiments where sustained release of the drug is desirable, the peak plasma concentrations (e.g., of therapeutic) may be maintained for 6-14 hours, e.g., for 6-12 or 6-10 hours. In other embodiments where immediate release of the drug is desirable, the peak plasma concentration (e.g., of therapeutic) may be maintained for, e.g., 30 minutes.

The frequency of treatment may also vary. The subject can be treated one or more times per day with therapeutic (e.g., once, twice, three, four or more times) or every so-many hours (e.g., about every 2, 4, 6, 8, 12, or 24 hours). Preferably, the pharmaceutical composition is administered 1 or 2 times per 24 hours. The time course of treatment may be of varying duration, e.g., for two, three, four, five, six, seven, eight, nine, ten or more days. For example, the treatment can be twice a day for three days, twice a day for seven days, twice a day for ten days. Treatment cycles can be repeated at intervals, for example weekly, bimonthly or monthly, which are separated by periods in which no treatment is given. The treatment can be a single treatment or can last as long as the life span of the subject (e.g., many years).

Kits

Any of the pharmaceutical compositions of the invention described herein can be used together with a set of instructions, i.e., to form a kit. The kit may include instructions for use of the pharmaceutical compositions as a therapy as described herein. For example, the instructions may provide dosing and therapeutic regimes for use of the compounds of the invention to reduce symptoms and/or underlying cause of the foam cell associated disease.

Wherefore we claim:

1. A method of treating a foam cell associated disease comprising:
administering a pharmaceutical composition including a therapeutically effective amount of a first therapeutic;
wherein the first therapeutic is one of an extract of Artemisia dracunculus (Russian Tarragon) leaves, an extract of Melia azedarach (Chinaberry) leaves, an extract of Saururus cernuus (Lizard's Tail) Roots, and an extract of Sambucus Canadensis (Elderberry) leaves, or pharmacologically acceptable salts, solvates, esters, amides, clathrates, stereoisomers, enantiomers, prodrugs or analogs thereof, or a combination thereof.

2. The method of claim 1 wherein the foam cell associated disease is one of kidney disease, cancer, metabolic syndrome (MetS), atherosclerosis, cardiovascular disease (CVD), tuberculosis, Parkinson's disease, a neurological disorder, and a disease where lipid droplet formation and autophagic flux play a role, or a pre-disease state thereof.

3. The method of claim 1 wherein the first therapeutic includes both an extract of Russian Tarragon leaves and an extract of Chinaberry leaves or pharmacologically acceptable salts, solvates, esters, amides, clathrates, stereoisomers, enantiomers, prodrugs or analogs thereof, or a combination thereof.

4. The method of claim 1 wherein the pharmaceutical composition further includes a therapeutically effective amount of a second therapeutic, distinct from the first therapeutic.

5. The method of claim 4 wherein the second therapeutic is a polyphenols.

6. The method of claim 5 wherein the polyphenol is a flavonoid.

7. The method of claim 4 wherein the second therapeutic is one of epigallocatechin-3-gallate (EGCG), luteolin, quercetin, and ellagic acid.

8. A method of treating an inflammation associated disease comprising:
administering a pharmaceutical composition including a therapeutically effective amount of a first therapeutic;
wherein the first therapeutic is one of an extract of Artemisia dracunculus (Russian Tarragon) leaves, an extract of Melia azedarach (Chinaberry) leaves, an extract of Saururus cernuus (Lizard's Tail) Roots, and an extract of Sambucus Canadensis (Elderberry) leaves, or pharmacologically acceptable salts, solvates, esters, amides, clathrates, stereoisomers, enantiomers, prodrugs or analogs thereof, or a combination thereof.

9. The method of claim 8 wherein the pharmaceutical composition further comprises a therapeutically effective amount of a second therapeutic distinct from the first therapeutic and wherein the second therapeutic is an anti-inflammation therapeutic.

10. The method of claim 8 wherein the inflammation associated disease is one of asthma, arthritis, Crohn's disease, Alzheimer's disease, cancer, cardiovascular disease, diabetes, high blood pressure, high cholesterol levels, Parkinson's disease, metabolic syndrome (MetS), atherosclerosis, or a pre-disease state thereof.

11. A therapeutic product comprising:
a first pharmaceutically active agent being one of one of an extract of Artemisia dracunculus (Russian Tarragon) leaves, an extract of Melia azedarach (Chinaberry) leaves, an extract of Saururus cernuus (Lizard's Tail) Roots, and an extract of Sambucus Canadensis (Elderberry) leaves, or pharmacologically acceptable salts, solvates, esters, amides, clathrates, stereoisomers, enantiomers, prodrugs or analogs thereof, or a combination thereof; and
a second pharmaceutically active agent being one of one of an extract of Russian Tarragon leaves, an extract of Chinaberry leaves, an extract of Lizard's Tail Roots, an extract of Elderberry leaves, and a polyphenol, or pharmacologically acceptable salts, solvates, esters, amides, clathrates, stereoisomers, enantiomers, prodrugs or analogs thereof, or a combination thereof;
wherein the first pharmaceutically active agent is chemically distinct from the second pharmaceutically active agent.

12. The therapeutic product of claim 11 wherein the first pharmaceutically active agent is extract of Russian Tarragon leaves or a pharmacologically acceptable salt, solvate, ester, amide, clathrate, stereoisomer, enantiomer, prodrug or analog thereof, or a combination thereof.

13. The therapeutic product of claim 11 wherein the first pharmaceutically active agent is on of an extract of Russian Tarragon leaves a pharmacologically acceptable salt, solvate, ester, amide, clathrate, stereoisomer, enantiomer, prodrug or analog thereof, or a combination thereof and the second pharmaceutically active agent is one of an extract of Chinaberry leaves a pharmacologically acceptable salt, solvate, ester, amide, clathrate, stereoisomer, enantiomer, prodrug or analog thereof, or a combination thereof.

14. The therapeutic product of claim 11 wherein the second pharmaceutically active agent is one of a polyphenol, a pharmacologically acceptable salt, solvate, ester, amide, clathrate, stereoisomer, enantiomer, prodrug or analog thereof, or a combination thereof.

15. The therapeutic product of claim 11 wherein the second pharmaceutically active agent is one of a flavonoid, a pharmacologically acceptable salt, solvate, ester, amide, clathrate, stereoisomer, enantiomer, prodrug or analog thereof, or a combination thereof.

16. The therapeutic product of claim 11 wherein the second pharmaceutically active agent is one of pigallocatechin-3-gallate (EGCG), luteolin, quercetin, ellagic acid, pharmacologically acceptable salts, solvates, esters, amides, clathrates, stereoisomers, enantiomers, prodrugs or analogs thereof, or a combination thereof.

17. The therapeutic product of claim 11 further comprising a third pharmaceutically active agent being one of one of an extract of Russian Tarragon leaves, an extract of Chinaberry leaves, an extract of Lizard's Tail Roots, an extract of Elderberry leaves, and a polyphenol, or pharmacologically acceptable salts, solvates, esters, amides, clathrates, stereoisomers, enantiomers, prodrugs or analogs thereof, or a combination thereof.

18. The therapeutic product of claim 17 wherein the first, the second, and the third pharmaceutically active agents are each extracts from different plants, or pharmacologically acceptable salts, solvates, esters, amides, clathrates, stereoisomers, enantiomers, prodrugs or analogs thereof, or a combination thereof.

19. The therapeutic product of claim 17 wherein the first and the second pharmaceutically active agents are each extracts from different plants, or pharmacologically acceptable salts, solvates, esters, amides, clathrates, stereoisomers, enantiomers, prodrugs or analogs thereof, or a combination thereof, and third pharmaceutically active agent is a polyphenol or a pharmacologically acceptable salt, solvate, ester, amide, clathrate, stereoisomer, enantiomer, prodrug or analog thereof, or a combination thereof.

20. The therapeutic product of claim 17 wherein the first pharmaceutically active agent is an extract from a plant, or a pharmacologically acceptable salt, solvate, ester, amide, clathrate, stereoisomer, enantiomer, prodrug or analog thereof, or a combination thereof, and the second and the third pharmaceutically active agent is a polyphenol or pharmacologically acceptable salts, solvates, esters, amides, clathrates, stereoisomers, enantiomers, prodrugs or analogs thereof, or a combination thereof.
